(19) United States
(12) Patent Application Publication
(10) Pub. No.: US 2006/0115430 A1
(43) Pub. Date: Jun. 1, 2006

(54) Calcium challenge test for detecting calcium homeostasis disorders

(76) Inventor: Thomas L. Cantor, El Cajon, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/260,864

(22) Filed: Oct. 27, 2005

Related U.S. Application Data
(60) Provisional application No. 60/640,418, filed on Dec. 30, 2004.
(60) Provisional application No. 60/622,883, filed on Oct. 27, 2004.

(51) Int. Cl. A61K 49/00

(52) U.S. Cl. 424/9.1

(57) ABSTRACT

The invention provides simple methods for detecting disorders of a subject's calcium homeostasis. The methods include administering a calcium salt to the subject and observing the effect of this dose of calcium on calcium levels in the subject's bodily fluids and/or tissues. The methods are useful to detect certain calcium homeostasis disorders or a predisposition for such disorders including adynamic bone disease and soft tissue calcification disorders, which are difficult to detect by other methods.

### RELATED APPLICATIONS

This application claims benefit of priority to U.S. provisional application Ser. No. 60/622,883, filed on 27 Oct. 2004, and U.S. provisional application Ser. No. 60/640,418, filed on 30 Dec. 2004. The contents of each of these applications are incorporated herein by reference in their entirety.

### FIELD OF THE INVENTION

The present invention provides methods for determining whether a subject suffers from a disorder in the physiological processes related to absorption, transport, storage, mobilization, or excretion of calcium. It provides a method in which calcium is administered to a subject, and the effect of that calcium on the concentration of calcium or other analytes in the subject's tissues or bodily fluids is observed. Measurements of calcium levels in the subject's bodily fluids or tissues are then used to determine whether the subject has a calcium homeostasis disorder or a predisposition for such disorders. As 98% of the body's calcium is stored in the bones, which are primarily composed of hydroxyapatite, Ca₁₀(OH)₂(PO₄)₆, the bones play an essential role in buffering changes in serum calcium. Use of a calcium challenge enables one to assess the ability of the bones to buffer calcium.

The methods are especially useful for determining whether a subject suffers from adynamic bone disease, a condition in which the subject does not have the turnover of calcium and other bone constituents that is necessary for bone tissue to be healthy and capable of properly buffering the body's calcium supply. The methods are also useful for the detection of certain other conditions aside from nephrolithiasis, osteoporosis, osteomalacia and primary hyperparathyroidism that affect calcium utilization or of a predisposition for such conditions, which include arteriosclerosis and other forms of soft tissue calcification including metastatic calcification and calciphylaxis. The invention also provides a kit useful for performing the test methods and recording or reporting results, and a method of doing business that includes providing such kits to users, receiving and analyzing samples from the users, and communicating analytical results to the users.

### BACKGROUND OF THE INVENTION

Calcium homeostasis involves a complex interplay between absorption, transport, storage in bones, deposition in other tissues, and excretion. See, e.g., Stipanuk, Biochemical and Physiological Aspects of Human Nutrition, W B Saunders (2000), pg. 648. This homeostatic balance is regulated by the hormones calcitriol, parathyroid hormone (PTH), and calcitonin. Disorders in any of these processes or their regulation may result in abnormal calcium levels or adverse consequences such as metastatic calcification of soft tissues, as well as disorders related to the role calcium plays in neurotransmission. Id. at 646. Calcium levels are also associated with other conditions and with overall mortality rates, at least in some populations: it has been found that elevation of either serum calcium or “Ca×PO₄ Product” is correlated with increased overall risk of death in hemodialysis patients. G. A. Block, J. Am. Soc. Nephrol. August 2004, 15(8), 2208-18. See also S. K. Ganesh, et al., J. Am. Soc. Nephrol. 12, 2131-38 (2001). Indeed, a statistically significant increase in the relative risk of mortality is found among hemodialysis patients with either high calcium levels or low phosphorus levels, though the correlation to levels of intact parathyroid hormone (iPTH), which is the primary regulator of calcium levels, was much weaker and only became statistically significant when iPTH levels were low. Block, pg. 2216. Thus the detection of calcium homeostasis disorders is especially important for treating patients requiring chronic hemodialysis treatments.

Some calcium homeostasis disorders are readily detected: hypercalcemia can be detected directly by measuring the levels of calcium in bodily fluids, and advanced soft tissue calcification can be detected by X-ray or electron-beam computed tomography (CT). Other such conditions cannot be detected so easily. For example, a patient may suffer from adynamic bone disease while exhibiting normal levels of serum calcium. It has now been found that chronic dialysis patients with low bone turnover had virtually identical levels of serum calcium to those with normal bone turnover: in fact, calcium levels did not correlate with low, normal or high bone turnover in these patients, although patients with high bone turnover had slightly elevated serum phosphate levels. Similarly, serum calcium levels are not well correlated with arterial calcification, although such calcification has been shown to be associated with adynamic bone disease. London, et al., J. Am. Soc. Nephrol. 15, 1943-51 (2004). Thus static measurements of calcium and phosphate levels may not detect some calcium homeostasis disorders.

Spencer, et al., developed an elaborate calcium utilization measurement that provides a method to diagnose senile osteoporosis. J. Am. Geriatr. Soc. 2(1), 19-25 (1954). These investigators placed patients on a low calcium diet for several days, then administered a bolus of calcium gluconate (440 mg of calcium) while the patients were fasting. The patient's excretion of urinary calcium was quantitated the day before this bolus was administered, the day it was administered, and the day after. The difference between the individual's intake and excretion was defined as calcium utilization, and the utilization efficiency was the ratio of this amount to the amount of Ca administered. A healthy control subject was found to have a calcium utilization efficiency of about 71% under these conditions; a patient with osteoporosis had measurably lower efficiency. The authors suggest that the test is useful for diagnosis of osteoporosis, but provide little guidance on its actual use. Renier, et al., on the other hand, suggests that a calcium tolerance test where calcium is administered intravenously is diagnostically useful only for osteomalacia. Rev. Rhum. Mal. Osteoartic. 45(10), 521-28 (October 1978) (only the abstract was obtained). Like Spencer, this work appears to rely at least partly on measurements of urinary calcium.

Holla, et al., provide a more quantitative calcium tolerance test for diagnosing osteoporosis: they administered calcium intravenously (3.6 mg/kg body weight) and measured serum calcium levels following the injection. Acta Med. Acad. Scient. Hung. 35(1), 53-59 (1978). They found that subjects having osteoporosis returned to their baseline serum calcium levels more slowly than control subjects, and from a very small data set, indicated that a serum calcium level higher than 11.0 mg/dl measured 60 minutes after the injection was highly correlated with osteoporosis. However, they also stated that serum calcium levels had returned to normal for all other patients, including those with bone diseases other than osteoporosis. Milkov, et al., appears to agree that a calcium tolerance test is useful for an early diagnosis of osteoporosis, though they measured osteocalcin rather than calcium. “Serum osteocalcin level as a marker of the functional state of osteoblasts after oral calcium tolerance test”, Vutr. Boles. 27(3), 101-106 (1988) (only the abstract was obtained). Blahos, et al., appears to be more concerned with hyperthyroidism than hyperparathyroidism, but does examine calcium levels using a calcium tolerance test and observes that calcium levels were still above baseline two hours after administration of a dose of calcium. “The calcium tolerance test in thyrotoxicosis, Hashimoto's thyroiditis, and after total thyroidectomy”, Vnitr. Lek. 42(9), 597-601 (September 1996) (only the abstract was obtained). Blahos also noted elevated levels of markers for both bone formation (osteocalcin and bone alkaline phosphatases) and bone resorption (urinary pyridinoline and deoxypyridinoline) in patients with untreated hyperthyroidism.

Adynamic bone disease (ABD) is a condition that is characterized by low turnover of the materials, mainly minerals, which make up the bones. The majority of calcium in the body is stored in bone: there, calcium provides strength and rigidity to the bones and shields the delicate and critical stem cells in the bone marrow from harmful radiation, while it also provides a storage mechanism for excess calcium. That storage is not static, however; the calcium in bone is continually reabsorbed and replaced, in a process that provides a buffer for the body's calcium supply, ensuring that calcium is available for the rest of the body when needed. Disorders which inhibit both resorption and deposition of bone result in low turnover of the minerals in bone, and are referred to as low bone remodeling or low bone turnover; this may result in osteomalacia (softening of the bones due to demineralization) or adynamic bone disease (ABD).

ABD is primarily a problem for patients undergoing chronic kidney dialysis. Currently half of patients undergoing dialysis suffer from adynamic bone disease. The condition has only been recognized recently, and was initially attributed to the aluminum salts (mainly aluminum hydroxide) given to patients with advanced renal failure in order to manage phosphate overload. Once the use of aluminum salts to reduce phosphate overload was curtailed, however, the incidence of recognized ABD did not decline. H. H. Malluche and M. C. Monier-Faugere, Kidney Int. Suppl. 38, S62-67 (October 1992). See also D. J. Sherrard, et al., “The spectrum of bone disease in end-stage renal failure—An evolving disorder,” Kidney Internat'l, 43, 436-42 (1993), which describes it as an ‘aplastic’ bone disorder that was little recognized in earlier studies and whose incidence was not correlated to aluminum levels in kidney patients. Thus ABD is generally not attributable to these aluminum salts, and its etiology remains unclear.

Sherrard distinguishes the aplastic condition now referred to as ABD from other bone disorders based partly on the osteoid area as a percentage of bone surface: the normal range for this parameter was reported as 1-7%, and a diagnosis of osteomalacia was made if the osteoid area exceeded 15%. Fibrosis as a percentage of bone surface area was also used: if fibrosis area exceeded 0.5%, the patient was diagnosed as having osteitis fibrosa. Aplastic disorder was diagnosed only where both osteoid and fibrosis areas were within normal ranges, and bone formation rate was below the normal range of 108-500 μm²/mm² tissue area/day. Where both osteoid and fibrosis were within normal range but some lesion was detected, the subject was classified as having “mild” lesion; and if the subject had abnormal levels of both osteoid and fibrosis areas, the lesion was classified as “mixed”.

Using the same classification system as that used by Sherrard, it has now been shown that the duration of dialysis does not seem to affect the distribution of bone disorders in dialysis patients. Overall, patients just starting on dialysis showed about the same distribution of bone status as patients on long-term maintenance. However, those patients having chronic glomerulonephritis showed a tendency for bone turnover to decrease over time, while those having diabetes mellitus showed the opposite trend. Thus among kidney dialysis patients, those experiencing chronic glomerulonephritis may be at greater risk for ABD and related calcium homeostasis disorders.

It has also been found that patients having ABD presented with serum calcium levels at or below normal when first placed on dialysis, but their serum calcium levels increased when they were placed on long-term maintenance. This suggests that such patients may gradually lose the ability to cope with excess calcium, possibly due to the long-term challenge of utilizing excess calcium. Thus a patient on dialysis may need to be monitored for development of calcium homeostasis disorders.

Victims of ABD may be asymptomatic. However, they suffer from reduced ability to mobilize calcium from bones when needed or to store calcium in bone efficiently when excess calcium is present in the blood. The storage deficiency may result in undesirable deposition of calcium in other tissues when calcium levels in the blood are high, and may also interfere with the ability of the bones to repair microscopic damage. Arterial calcification also appears to be associated with adynamic bone disease. See G. M. London, et al., J. Am. Soc. Nephrol. 15, 1943-51 (2004). Over time, ABD may weaken bones, too: according to the National Kidney Foundation, this may contribute to a four-fold increase in the incidence of hip fractures among dialysis patients. See the internet page on the world wide web at kidney.org/professionals/kdoqi/guidelines_bone/Guide13C.htm. Furthermore, the reduced ability to mobilize calcium from bones means that victims of ABD require dietary calcium sufficient to meet their daily needs, so they cannot rely on a low-calcium diet to avoid the calcium overload that leads to undesirable calcification of tissues other than bone.

The balance between the different processes for the absorption, utilization, and excretion of calcium can mask deficiencies in any one process: if calcium storage is poor in a particular subject, for example, efficient excretion may result in the subject maintaining ‘normal’ levels of calcium circulating in his or her blood. Likewise, when both deposition of calcium into bone and resorption of calcium from bone are curtailed in ABD, the net calcium level may appear unaffected due to efficient elimination of excess dietary calcium, even though the dynamic storage and deposition processes are strongly inhibited. It has thus been found that serum calcium levels were similar in patients with low, normal, or high bone turnover; furthermore, parathyroid hormone (PTH) levels have not yet been shown to predict ABD. Monier-Faugere, et al., Kidney International, 60, 1460-68 (2001). Detection of ABD therefore relies largely on intrusive bone biopsy test methods. Since ABD can affect how other conditions commonly found in dialysis patients should be treated, its diagnosis is especially important in that population. Similarly, a patient at risk for osteoporosis might be treated with calcium supplements and could maintain a normal serum calcium level, yet suffer from ABD. The excess calcium introduced by the calcium supplements may accelerate arterial calcification, since the normal mechanism for coping with excess calcium, deposition into bone tissue, is deficient in the ABD victim.

In addition to ABD and other bone disorders, calcium homeostasis disorders include conditions in which calcium-containing materials are deposited in soft tissues. While such disorders may accompany ABD or other bone diseases, they may also arise from other causes: excessive intestinal absorption of calcium, overdose of vitamin D or other substances that cause excess absorption or retention of calcium, renal failure preventing excretion of calcium when needed, or hyperparathyroidism, where excess parathyroid hormones (e.g., PTH₁₋₈₄) cause retention of excess calcium. For subjects having these disorders, simply measuring calcium levels may not be diagnostic: London (J. Am. Soc. Nephrol. 15, 1943-51 (2004)), for example, reported that calcium levels were not meaningfully correlated with the extent of arterial calcification in a group of end-stage renal disease (ESRD) patients.

Dialysis patients are particularly susceptible to some calcium homeostasis disorders including metastatic calcification and coronary artery calcification. See, e.g., Goodman, et al., New England J. Med. 342(20), 1478-83 (2000). Goodman found that dialysis patients over 20 years of age having ESRD frequently have some degree of arterial calcification (coronary artery calcification was found in 14 of 16 dialysis patients who were between 20 and 30 years of age). Furthermore, the extent of calcification increased markedly over time: the average calcification score for ten such subjects nearly doubled in less than two years.

Thus calcium homeostasis disorders may be difficult to diagnose by measuring static calcium levels, and there is no clear correlation of parathyroid hormone (PTH) level with ABD. See Monier-Faugere. Detection of ABD is especially important for dialysis patients, and currently requires relatively expensive and invasive tests that involve directly sampling the subject's bone (biopsy). And the prevalence of arterial calcification in renal patients coupled with the fact that such calcification can be exacerbated by treatments commonly administered to dialysis patients (see, e.g., Goodman, showing that the extent of coronary artery calcification was correlated with the amount of calcium administered to the patient as a phosphate binder) demonstrates the importance of recognizing a tendency to develop such calcium homeostasis disorders. Thus more convenient and less costly methods are needed for the detection of ABD, metastatic calcification, and a predisposition or tendency for these and other calcium homeostasis disorders. The present invention provides such methods.

### SUMMARY OF THE INVENTION

The present invention provides a method for detecting certain calcium homeostasis disorders that are not readily observed by current diagnostic tests, which usually measure levels of calcium in a fasting subject's blood or urine. It also allows detection of a predisposition for such calcium homeostasis disorders as ABD and metastatic calcification. The invention is especially suitable for the detection of adynamic bone disease and for detection of a predisposition for calcification of soft tissues, including disorders such as arteriosclerosis, metastatic calcification, and calciphylaxis.

The methods of the invention involve administering a calcium salt to a subject to be tested, and then measuring how the dose of calcium affects levels of calcium or of other analytes in the bodily fluids or tissues of the subject. Optionally, a subject receiving a calcium salt orally is also administered an amount of vitamin D that is effective to enhance absorption of the calcium salt in the subject's gastrointestinal system. The invention includes methods where other analytes such as magnesium, phosphate, or parathyroid hormone (PTH) or fragments of PTH are measured in the subject's bodily fluids or tissues in addition to or instead of measuring calcium levels for detecting a calcium homeostasis disorder. The method is applicable to calcium homeostasis disorders other than nephrolithiasis (kidney stone formation), osteoporosis, osteomalacia and primary hyperparathyroidism, each of which can be detected by other methods.

In one aspect of the invention, calcium levels are measured in the subject's blood, serum, saliva, or urine after a calcium salt has been administered to the subject. Frequently, the level of calcium is measured in the subject's blood, and often it is measured both before and after administering the calcium salt. Two or more measurements of the calcium level in the subject's bodily fluids after administration of the calcium salt may advantageously be obtained.

The calcium salts useful for the invention include all calcium salts that may safely be administered to a subject to be tested; examples include calcium carbonate, calcium chloride, calcium citrate, calcium tartrate, calcium hydroxide, calcium acetate, calcium nitrate, calcium succinate, an alkylsulfonate or arylsulfonate salt of calcium, calcium gluconate, calcium gluceptate, calcium glubionate, calcium lactate, calcium glycerophosphate, and calcium glucuronate, as well as combinations of these. The calcium salt may be administered orally or intravenously, and may be administered all at once or over time. If it is to be administered orally, milk may be used as a suitable vehicle for delivery of calcium. For a patient undergoing hemodialysis or other medical procedures, the administration of a calcium salt and the measurement of calcium levels in the subject's blood may be done over time, such as while the subject is undergoing a dialysis treatment.

A subject suffering from a calcium homeostasis disorder may show a reduced ability to recover to the calcium level that is normal for that subject or similar subjects following administration of the calcium salt, or may exhibit a greater than normal increase in calcium level during the time period following administration of the calcium salt, or may take longer to return to the subject's normal calcium level following administration of the calcium salt. The invention includes measuring any of these parameters for use in diagnosing calcium homeostasis disorders.

In some embodiments, the bodily fluid used is blood or serum, and the concentration of calcium is referred to as the subject's serum calcium level. Where a subject has a serum calcium level higher than about 10 mg/dl after administration of a calcium salt, a calcium homeostasis disorder may be present. Where a subject has an increase in serum calcium level of more than about 1.0 mg/dl following administration of a calcium salt, a calcium homeostasis disorder may be present. Where levels of other analytes besides calcium are measured, the effect of the calcium challenge test on levels of those analytes may also be used to diagnose the presence or absence of a calcium homeostasis disorder. The method is useful for the detection of ABD, soft tissue metastatic calcification, or a predisposition for ABD or soft tissue metastatic calcification such as arteriosclerosis caused or exacerbated by inadequate calcium homeostatic processes. The invention is especially useful for diagnosing a human subject, and is often used for a patient having a kidney disorder, especially one undergoing dialysis treatment or an end-stage renal disease patient.

The invention also provides a kit that includes some or all of the following: a calcium salt, apparatus to facilitate collecting, storing or shipping samples of bodily fluids, and means to measure the concentration of one or more analytes, as well as instructions and forms useful for recording, labeling, shipping, or reporting the samples and/or results of the test in accordance with the present invention.

The invention further provides a method of doing business which includes some or all of the following: providing the above kit to a user, receiving samples from the user, measuring the levels of calcium and optionally other analytes in samples provided by the user, and communicating the results of those measurements to the user. Often the user will be a physician who is treating a dialysis patient.

### DETAILED DESCRIPTION OF THE INVENTION

Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, the term “calcium homeostasis disorder” is intended to include any condition other than kidney stone formation (nephrolithiasis), osteomalacia, osteoporosis, or subtle primary hyperparathyroidism in which calcium absorption, utilization, storage, or deposition is substantially affected. The term includes adynamic bone disease and other conditions in which the rate of deposition of calcium into bone is affected. It also includes soft tissue calcification conditions, including metastatic calcification, calciphylaxis, and arterial diseases in which calcium is incorporated into deposits in arteries, such as arteriosclerosis. It also includes conditions in which the kidneys are unable to eliminate calcium at a sufficient rate.

As used herein, “disease or disorder” refers to a pathological condition in an organism resulting from, e.g., infection, injury or genetic defect, and characterized by identifiable symptoms.

As used herein, the term “subject” is not limited to a specific species or sample type. For example, the term “subject” may refer to a patient, and frequently a human patient. However, this term is not limited to humans and thus encompasses a variety of mammalian species.

As used herein, “afflicted” as it relates to a disease or disorder refers to a subject having or directly affected by the designated disease or disorder.

As used herein the term “sample” refers to anything which may contain an analyte for which an analyte assay is desired. The sample may be a biological sample, such as a bodily fluid or a biological tissue. Examples of samples that are bodily fluids include urine, blood, plasma, serum, saliva, semen, stool, sputum, cerebral spinal fluid, tears, mucus, amniotic fluid or the like. Biological tissues are aggregates of cells, usually of a particular kind, together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, the term ‘parathyroid hormone or fragments thereof’, or equivalently the term ‘PTH or fragments of PTH’ refers to the intact human parathyroid hormone (iPTH) or substantial fragments thereof that affect calcium homeostasis. Such fragments include, for example, any form of human parathyroid hormone that has been truncated by removal of from one to seven of the first amino acids of the 84-amino acid polypeptide known as PTH. The form where the first six amino acids have been removed, referred to as 7-84 PTH (or PTH₇₋₈₄), is particularly included because it is known to be important in calcium homeostasis, as is iPTH.

As used herein, the term “metastatic calcification” refers to the physiological deposition of any calcium-containing material into otherwise healthy tissue other than bone. It is distinguished from “dystrophic calcification”, which involves the deposition of calcium-containing material into damaged or necrotic tissues other than bone. Both types of calcification are potentially exacerbated by elevated levels of calcium, although metastatic calcification may be mediated by processes that regulate calcium levels (see London, at 1943: “recent studies have shown that AC (arterial calcification) is a regulated process . . . . In addition, evidence indicates that many proteins involved in bone metabolism can be expressed in arterial tissue . . . ”), while dystrophic calcification may occur as a response to inflammation even when no calcium homeostasis disorder is present.

As used herein, the term “predisposition” refers to a statistically significant increase in the risk of a certain event or disorder in a particular subject relative to an average individual. Such increased risk may arise from other medical conditions the subject is experiencing, from treatments used to control other medical conditions, from genetic factors, dietary or other lifestyle factors, or from a combination of these.

As used herein, the term “calcium salt” refers to any calcium salt that can safely be administered to a test subject, and includes chloride, hydroxide, carbonate, citrate, gluconate, glucuronate, glubionate, lactate, glycerophosphate, acetate, nitrate, sulfate, alkylsulfonate, arylsulfonate, tartrate, and other organic and inorganic salts of calcium that have adequate water solubility to be absorbed into or redistributed within the subject's body and low enough toxicity to be useful for a medical diagnostic test procedure. The term includes mixtures such as milk, which may be used to deliver calcium orally, as well as readily available forms of calcium such as calcium supplements and over-the-counter medications such as TUMS®, an antacid product which is manufactured by GlaxoSmithKline and which contains calcium carbonate as its active ingredient.

The Invention

The methods of the invention are useful to provide diagnostic information for subjects who may be at risk for calcium homeostasis disorders. Patients suffering from kidney disease are often appropriate subjects for the methods of this invention. Since calcium homeostasis disorders are often found in dialysis patients, the methods are particularly useful for diagnosing or monitoring such patients.

In one aspect, the invention provides a method for detecting certain calcium homeostasis disorders such as ABD by administering a dose of a calcium salt to the subject to be tested and measuring calcium levels in the subject's blood, saliva, or urine or other tissue or bodily fluid. In a preferred embodiment, the bodily fluid or tissue is either blood or urine or saliva; blood is sometimes preferred, and the concentration of calcium in the blood is often referred to as the subject's serum calcium level, which is typically expressed in milligrams of calcium per deciliter of blood (mg/dl).

The subject is often one suspected to have or be at risk for a calcium homeostasis disorder; thus the subject may be someone with a kidney disease or renal failure, especially someone undergoing kidney dialysis treatments. Patients having chronic glomerulonephritis are sometimes appropriate subjects. Alternatively, the subject may be someone at risk for such disorders based on statistical considerations of various factors such as gender, age, height, weight, race, or diet, or the subject may be someone suffering from other conditions that are associated with one or more calcium homeostasis disorders, or someone undergoing a medical treatment that is known or suspected to cause a calcium homeostasis disorder.

It is sometimes desirable to control or monitor the dietary intake of calcium by the subject to be tested prior to administration of a calcium challenge test of the invention to ensure that an appropriate ‘normal’ or baseline level of calcium is reached. Thus prior to administering the calcium challenge test, the subject to be tested may optionally be directed to discontinue ingestion of calcium supplements or other calcium containing medications, and optionally may be placed on a controlled or restricted diet to limit intake of calcium, phosphate, or other materials that could influence the test results. Usage of medications that may affect the test or its interpretation may also be adjusted before and during the test; for example, dosages of vitamin D that the subject would otherwise receive may be modified or discontinued in preparation for the test. For a subject on dialysis, the dialysate composition to be used by the subject may be adjusted as appropriate in preparation for the test.

In some embodiments, the calcium salt may be administered orally; in others, it is administered intravenously. Optionally, it may be administered during a dialysis treatment as a component of the dialysate or by introducing it into the subject's blood via the dialysis system. When the salt is administered orally, the subject may be asked to ingest a tablet or tablets containing a calcium salt such as, for example, calcium carbonate, or the subject may be asked to ingest a solution of a calcium salt in water, or an appropriate quantity of milk, for example, which contains about 300 mg of calcium per cup. When the calcium salt is administered orally, the subject may also be treated with an amount of vitamin D that is effective to enhance absorption of the calcium salt. The vitamin D may be administered along with the calcium salt, for example by administering the salt in the form of a dietary supplement containing both calcium carbonate and vitamin D; or the vitamin D may be administered separately, either orally or by injection. When the calcium salt is administered intravenously, it may be delivered in one bolus or it may be delivered in several smaller portions over a period of time, or it may be delivered continuously by an IV drip. Optionally, it may be incorporated into the composition of the dialysate used in a dialysis process or introduced directly into the subject's blood during dialysis.

Calcium salts suitable for the invention include calcium carbonate, calcium nitrate, calcium chloride, calcium hydroxide, calcium citrate, calcium acetate, calcium tartrate, calcium succinate, calcium gluconate, calcium glucuronate, calcium glubionate, calcium lactate, calcium glycerophosphate, and other organic and inorganic salts of calcium. The calcium salt should have sufficient water solubility to allow it to redistribute in the body of the subject to be tested, and should have low enough toxicity to minimize adverse effects on the subject. Calcium carbonate is often preferred, and where the calcium salt is administered as an aqueous solution, calcium chloride is sometimes preferred. Mixtures of the above salts, including milk or compositions such as dietary supplements containing a calcium salt plus vitamin D, may also be utilized.

The amount of the calcium salt to be used is readily determined by one of ordinary skill in the art, and the amount will vary depending on the age, gender and weight of the subject to be tested. The amount may be from 50 mg to 5000 mg depending upon the parameters of the test and the physiology of the subject. Typically, at least 250 mg of calcium carbonate or a calcium salt of similar molecular weight is needed if the calcium salt is administered orally. A larger amount may be advantageous to ensure that a measurable response is observed; thus 350 mg or 500 mg or 750 mg or 1000 mg of the calcium salt may be administered orally, or 1500 mg or 2000 mg or 2500 mg may be used when appropriate. If the calcium salt is administered intravenously, a smaller amount such as 50 mg or 100 mg may be adequate, since intravenous administration bypasses the intestinal absorption process and accelerates entry of the calcium into the subject's blood stream. Nevertheless, a larger amount may be used for intravenous administration as well, and amounts of 150 mg or 200 mg or 350 mg or 500 mg or more may be administered intravenously in order to produce a useful diagnostic result. The amount of calcium salt is selected to ensure that a useful diagnostic result is obtained, so an appropriate amount may easily be determined with minimal experimentation. The amount of calcium in a dialysate will typically vary from 1.2 to 4.0 meq/L, but higher concentrations of calcium may be used if calcium is to be delivered through the dialysate.

In one aspect, the method of the invention is used to detect adynamic bone disease (ABD), or slow bone turnover. In one embodiment, it is used to determine whether the subject is predisposed to develop ABD.

In another aspect, it is used to detect the occurrence of or a predisposition for a calcium homeostasis disorder that results in deposition of calcium, such as soft tissue calcification. In still another embodiment, the method is used to detect a tendency for a subject to develop arteriosclerosis or soft tissue metastatic calcification.

In some embodiments, the subject's calcium level is measured before administration of the calcium salt, which is referred to as the subject's normal level, and is then measured again at one time point or more than one time point after administration of the calcium salt. Measurements made after administration of the calcium salt are ordinarily made within 24 hours after administering the calcium salt, preferably within 8 to 12 hours, and more commonly within 0.1 to 6 hours after the calcium salt is administered. In one embodiment, at least one measurement is made within about one hour after the salt is administered. In another embodiment, the subject's serum calcium level is measured before administration of the calcium salt and it is measured again at multiple time points afterwards, for example, every 10 minutes or every 20 minutes after administration of the calcium salt for the first hour and every 30 minutes or every 60 minutes thereafter until the subject's serum calcium level returns approximately to its normal level or a relatively stable level. These measurements after administering the calcium salt may be made at precisely defined time intervals, or they may be made periodically as convenient over the first 4, 6, 8, or 12 hours following administration of the calcium salt. A graph of calcium level changes over time may then be generated and used to interpolate or extrapolate so that appropriate parameters can be obtained for comparison to statistically determined normal calcium levels or to an average or subject-specific baseline, for example.

In other embodiments, the level of calcium in the subject's tissue or bodily fluid is measured two or more times after administration of the calcium salt, and the user can determine a rate at which the subject's calctum level returns to the normal level for the subject. Either a calcium level from the subject's tissue or bodily fluid which is measured at a point in time after administration of the calcium salt, or the rate at which the subject's calcium level returns to the level that is normal for that subject, or the length of time required for the subject's serum calcium level to return to a normal or average level may be compared to the corresponding values obtained from healthy individuals in order to diagnose whether the subject has a calcium homeostasis disorder or a predisposition for such disorder.

In some embodiments, the concentration of calcium in the tissue or bodily fluid of the subject to be tested is measured before the calcium salt is administered. In such embodiments, the concentration of calcium measured before the calcium salt is administered provides a baseline or ‘normal’ value for comparison to later measurements of calcium levels in the subject's tissue or fluid; alternatively, the concentration of calcium measured before the calcium salt is administered may be used to normalize the subject's calcium level to an average calcium level determined by measuring calcium levels in similar tissue or bodily fluid samples from healthy individuals. The concentration of calcium measured before the calcium salt is administered may be used to adjust or correct measurements of calcium levels made after the administration of the calcium salt so that the subject's response to the administration of the calcium salt can be compared to responses seen in healthy individuals.

In each case, the calcium level measured after administration of the calcium salt is compared to an appropriate baseline value for healthy individuals or the particular subject. A calcium level increase that is significantly above the normal increase produced by the amount of calcium administered, allowing for the subject's gender, age and weight, is indicative of a calcium homeostasis disorder. A rate of return to normal calcium levels that is significantly lower than the average rate of return to normal, allowing for the subject's gender, age and weight, is indicative of a calcium homeostasis disorder. Or a length of time required for recovery to the subject's normal calcium level that is significantly above the average length of time required to recover to normal, allowing for the subject's gender, age and weight, is indicative of a calcium homeostasis disorder.

In still other embodiments, the calcium salt may be delivered over time such as by an intravenous drip, and the subject's calcium level may be measured during or after completion of such calcium salt administration. Alternatively, where the subject is undergoing kidney dialysis treatment, the calcium salt may be administered and calcium measurements may be made prior to, after, or during the course of a dialysis treatment. Where the calcium salt is administered over time, a graph or profile of the subject's calcium level may be generated, and this graph or profile may be compared to an average profile for healthy individuals of similar characteristics in order to diagnose the subject's condition.

In a preferred embodiment of the invention, the subject is human. It is reported that the average serum calcium level for humans is around 9.0 mg/dl. In some embodiments, a human subject is thus diagnosed as having a calcium homeostasis disorder if the subject has a serum calcium level higher than about 9.5 or higher than 10 or higher than 11 mg/dl at some time point after administration of the calcium salt, such as 30 minutes or 60 minutes after, or at the peak level attained following the calcium salt administration. In other embodiments, the diagnosis is based on the extent to which the subject's calcium level is increased after the calcium salt is administered, and a subject may be diagnosed as having a calcium homeostasis disorder if the subject's serum calcium level is higher by 0.5 mg/dl or by 1.0 mg/dl or by 1.5 mg/dl at at least one time point after the calcium salt is administered than it was prior to administration of the calcium salt. In still other embodiments, the diagnosis is based in whole or in part on the effects that the calcium salt produces on the levels of other analytes, such as phosphate, magnesium, or PTH or fragments of PTH.

In one aspect, the invention thus provides a method for detecting adynamic bone disease in a subject by:

(1) determining a baseline value for calcium levels in healthy individuals receiving a specified amount of a calcium salt;

(2) administering a similar amount of a calcium salt to the subject to be diagnosed, which amount may be adjusted for the subject's age, gender, and/or weight;

(3) measuring a calcium level in a sample obtained from the subject to be diagnosed; and

(4) comparing the subject's calcium level to that of healthy individuals receiving similar calcium salt dosages, or to the subject's calcium level before the salt was administered.

Optionally, a normal or baseline calcium level for a subject to be diagnosed may be determined by measuring the calcium level in the subject's tissue or bodily fluid before the calcium salt is administered, and this value may be used to normalize the calcium level measured for the subject's tissue or bodily fluid after the calcium salt is administered to the subject, in order for the subject's calcium level to be compared directly with those of healthy individuals. Also optionally, the subject's calcium level may be measured at multiple time points to provide a profile or a rate of change in calcium levels, which may also be compared to appropriate norms to arrive at a diagnosis.

In another aspect, the above method is used to detect soft tissue metastatic calcification or arteriosclerosis in a subject, or to determine whether the subject is predisposed to develop such disorders. Where a subject's calcium level is increased more than normal by the calcium salt administered, or where the subject's calcium level takes longer to return to its normal level after the calcium salt is administered, or where the subject's calcium level returns to normal at a slower rate than the average rate, the subject may be diagnosed as being at risk for soft tissue calcification disorders or the subject may have a soft tissue calcification disorder.

In certain embodiments of the invention, the concentration of at least one analyte besides calcium, such as magnesium, phosphate, or PTH or fragments thereof, is measured in a sample of a bodily fluid from the subject. Methods for measuring levels of calcium, magnesium and phosphate are well known in the art. Methods for measuring levels of PTH or fragments of PTH are described in U.S. Pat. Nos. 6,689,566 and 6,743,590 and U.S. patent application Ser. No. 10/617,489, which was filed on 10 Jul. 2003, each of which is incorporated herein by reference in its entirety.

In yet another aspect, the invention provides a kit that is useful for administering the method of the invention. The kit includes some or all of the following: a calcium salt; means for collecting a bodily fluid or tissue sample; means for preserving or preparing the sample for storage, shipping or analysis; means for measuring the concentration of at least one analyte in the tissue or bodily fluid sample; forms for recording or reporting information about the sample and/or results of the analysis; vouchers to expedite processing of samples through an analytical lab; and instructions on administering the test procedures, and optionally also instructions for interpreting the results of the calcium challenge test.

Means for collecting a bodily fluid or tissue sample will vary depending on the sample required and depending on by whom the test is to be administered; typically, such means would include a container to hold the sample and, for example, a hypodermic syringe plus needle for withdrawing a sample of blood or serum. Means for measuring the concentration of an analyte are well known for many of the analytes of interest. For example, an antibody-based affinity method such as a sandwich assay may be usefully incorporated into the kits of the invention to measure PTH or fragments thereof. Alternatively, the means for measuring the concentration of an analyte may comprise means to deliver the sample containing the analyte to an analytical laboratory for analysis, which could include sample containers, storage instructions and shipping instructions.

In another aspect, the invention provides a business method that includes providing the above described kit to a user along with instructions for administering the test and collecting suitable samples for analysis. The business method further includes receiving samples from a user of the kit, measuring the levels of calcium and/or other analytes in samples collected with the kit, and communicating the results of those measurements to the user.

### EXAMPLES

The following examples illustrate the methods of the present invention. However, they only exemplify certain embodiments and are not intended to limit the invention.

### Example 1

A subject at risk for a calcium homeostasis disorder is identified. A blood sample is taken and the subject's serum calcium level is measured to establish a baseline or normal serum calcium level for the particular subject. The subject is asked to ingest a calcium salt. For example, the subject may ingest 500 to 2500 mg of calcium carbonate in the form of tablets such as Tums®; alternatively, the subject may drink an appropriate quantity of milk, which contains approximately 300 mg of calcium per cup. Following ingestion of the calcium salt, additional blood samples are taken at 30 minute intervals for two hours and then hourly for the next six hours. An additional sample is taken 24 hours after administration of the calcium salt. Serum calcium levels are measured for each blood sample taken from the subject.

The subject's serum calcium level at each time point, which may be adjusted if the subject's normal calcium level differs significantly from the average individual's ‘normal’ calcium level, is compared to that observed for average individuals of similar age and weight. If the subject's serum calcium level is significantly higher than that of an average person, or if the subject's rate of return to his or her normal calcium level is significantly slower than that of an average person, or if the length of time required for the subject's serum calcium level to return to normal is significantly longer than average, the subject is diagnosed as having a calcium homeostasis disorder or a predisposition for such disorder.

### Example 2

A subject at risk for a calcium homeostasis disorder is identified. A blood sample is taken and the subject's serum calcium level is measured to establish a baseline or normal serum calcium level for the particular subject. A solution containing 50 to 250 mg of a calcium salt, preferably calcium carbonate or calcium chloride or a mixture of the two, is injected into the subject intravenously. Following injection of the calcium salt, additional blood samples are taken at 30 minute intervals for two hours and then hourly for the next six hours. An additional sample is taken 24 hours after administration of the calcium salt. Serum calcium levels are measured for each blood sample taken from the subject, and the results are interpreted as described in Example 1 above.

### Example 3

A subject at risk for a calcium homeostasis disorder is identified. A blood sample and a urine sample are taken from the subject, and the subject's serum and urinary calcium levels are measured to establish baseline calcium levels for the particular subject. A solution containing 50 to 250 mg of a calcium salt, preferably calcium carbonate or calcium chloride or a mixture of the two, is injected into the subject intravenously. Following injection of the calcium salt, additional blood samples are taken at 30 minute intervals for two hours and then hourly for the next six hours. An additional sample is taken 24 hours after administration of the calcium salt. The subject's urine is also collected during the 24 hours following administration of the calcium salt. Serum calcium levels are measured for each blood sample taken from the subject, and the results are interpreted as described in Example 1. In addition, the amount of calcium excreted in the subject's urine within 24 hours after administration of the calcium salt is compared to that excreted by an average healthy individual undergoing the same diagnostic procedure. Excretion of a significantly lower amount of calcium by the subject relative to a healthy individual indicates the subject has a calcium homeostasis disorder or a predisposition for such disorders.

### Example 4

A kit is assembled for use by a physician in the diagnosis of a subject at risk for adynamic bone disease. The kit includes about 20 TUMS® tablets, each of which contains 500 mg of calcium carbonate. It also includes a set of Vacutainers® for collecting blood samples from the subject before and after the patient consumes a specific amount of calcium carbonate. The set of Vacutainers® includes:

2 red top (serum) 5 mL Vacutainers®, each having a tube for a blood sample and one for separated serum

2 lavender top (EDTA) 8 mL Vacutainers®, each having a tube for blood and one for separated plasma

One Vacutainer® of each color is labeled “Pre-Calcium Challenge Sample” and the other is labeled “Post-Calcium Challenge Sample”. Each label also provides space for recording patient identifying information. The kit also includes vouchers for two calcium level analyses and vouchers for two CAP™ and two Total PTH™ (tPTH) tests that allow determination of Accuratio™ values for use in conjunction with the calcium level measurements to diagnose the subject's bone status.

PTH Accuratio™—1-84 PTH/7-84 PTH ratio—is Scantibodies' new trademark for the state-of-the-art panel available at Scantibodies Clinical Lab. It is composed of the CAP™ 1-84 Whole PTH™ Assay value, the Total PTH™ (intact PTH) value and the calculated value of CIP™ (7-84 PTH). From these values, the ratio is assembled, giving physicians a more accurate, non-invasive way to determine bone status in renal and osteodystrophy patients, as well as a better way to monitor PTH levels.

Total PTH™—(tPTH) is Scantibodies improved version of the 2nd generation PTH test, which measures the sum of 1-84 PTH and 7-84 PTH. Total PTH values are more accurately determined with this newer test because of the highly specific antibody developed by Scantibodies for this measurement.

CAP™ (Whole PTH) Assay—is Scantibodies 1-84 Whole PTH assay newly developed with a highly sensitive and specific antibody to measure 1-84 PTH without the cross-reactivity to 7-84 PTH of the intact PTH assay. It is separate and distinct from the 2nd generation PTH assays, as well as the Bio-Intact PTH Assay, recently marketed by competitors. All 3rd generation PTH assays are NOT created equal.

The kit further includes instructions for the physician who will administer the test, including directions the physician should give to the subject to be tested. For diagnosing the presence or absence of ABD in a subject who is undergoing dialysis treatments, the instructions should tell the physician to do the following:

1. Direct the subject to discontinue taking any calcium or vitamin D supplements for one week prior to testing.

2. Before beginning dialysis (day one), draw one red Vacutainer® tube and one lavender Vacutainer® tube of blood from the subject, and label them as ‘Pre-Calcium Challenge Test’ samples and include patient identifying information. Then immediately centrifuge the samples and pipette the separated serum and plasma samples into the corresponding labeled transfer tubes. The serum and plasma samples should then be stored frozen at −20° C.

3. Direct the subject to take one TUMS tablet every two hours during the day for two days, then return for a second blood sample to be collected.

4. Before beginning dialysis on day three, draw one red Vacutainer® tube and one lavender Vacutainer® tube of blood from the subject, and label them as ‘Post-Calcium Challenge Test’ samples and include patient identifying information. Then immediately centrifuge the samples and pipet the separated serum and plasma samples into the corresponding labeled transfer tubes. The serum and plasma samples should then be stored frozen at −20° C.

5. Ship the two labeled serum-containing tubes and the two labeled plasma-containing tubes in Dry Ice, along with the four vouchers, to a Scantibodies clinical laboratory for analysis. The Scantibodies lab will test the samples and provide test results to the physician. The subject's response to the calcium challenge test will be compared to the typical responses seen for individuals not experiencing ABD and those with ABD to diagnose the subject's condition.

One of ordinary skill can appreciate that the present invention can incorporate any number or combination of the embodiments disclosed above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

I claim:

1. A method to detect a calcium homeostasis disorder or a predisposition for such disorder in a subject,
with the proviso that said disorder is not nephrolithiasis, osteomalacia, osteoporosis, or primary hyperparathyroidism,
said method comprising administering a calcium salt to the subject and subsequently collecting a sample of a bodily fluid from the subject, and
measuring the concentration of calcium in a bodily fluid from said subject,
whereby a calcium homeostasis disorder is diagnosed in said subject if the subject's calcium level after administration of said calcium salt is significantly higher than that of an average person, or if the subject's rate of return to his or her normal calcium level is significantly slower than that of an average person, or if the length of time required for the subject's serum calcium level to return to normal is significantly longer than average.

2. The method of claim 1, further comprising at least one of the following:
subtracting the calcium level measured in a bodily fluid taken from said subject before the administration of said calcium salt from a concentration measured in a sample of the bodily fluid taken from said subject after administration of said calcium salt to calculate the increase in the subject's calcium level, and comparing the increase in the subject's calcium level to that observed for an average healthy individual;
or calculating the rate at which the subject's calcium level returns to the subject's normal level, and comparing that rate to the corresponding rate for an average healthy individual;
or calculating the length of time required for the subject's calcium level to return to the subject's normal level, and comparing that length of time to the corresponding time for an average healthy individual.

3. The method of claim 1, wherein a subject having a serum calcium level above about 10 mg/dl following the administration of a calcium salt is diagnosed as having a calcium homeostasis disorder.

4. The method of claim 1, wherein said subject is a human.

5. The method of claim 1, wherein said bodily fluid is blood, serum, saliva, or plasma.

6. The method of claim 1, wherein said calcium salt is selected from the group consisting of calcium carbonate, calcium chloride, calcium citrate, calcium tartrate, calcium hydroxide, calcium acetate, calcium nitrate, calcium succinate, calcium lactate, calcium glubionate, calcium gluceptate, calcium glycerophosphate, an alkylsulfonate or arylsulfonate salt of calcium, calcium gluconate, and calcium glucuronate.

7. The method of claim 1, wherein the calcium salt is administered orally, and optionally an effective amount of vitamin D is administered in addition to the calcium salt.

8. The method of claim 1, wherein the calcium salt is administered intravenously.

9. The method of claim 1, wherein the calcium homeostasis disorder is one which affects calcium deposition.

10. The method of claim 1, wherein the calcium homeostasis disorder is adynamic bone disease.

11. The method of claim 1, wherein the calcium homeostasis disorder is a form of soft tissue calcification.

12. The method of claim 1, further comprising the step of measuring the concentration of calcium in the same tissue or bodily fluid of said subject prior to administering said calcium salt to said subject.

13. The method of claim 12, wherein the subject is diagnosed as having a calcium homeostasis disorder if the subject's serum calcium level after administration of the calcium salt is higher by more than about 1.0 mg/dl than the subject's serum calcium level was before administration of the calcium salt.

14. The method of claim 1, wherein the concentration of calcium is measured within about 24 hours after administering said calcium salt.

15. The method of claim 10, wherein the concentration of calcium is measured at two or more time points after administering said calcium salt.

16. The method of claim 1, wherein said subject has a kidney disorder or is undergoing kidney dialysis treatments.

17. The method of claim 16, where said subject has chronic glomerulonephritis.

18. The method of claim 5, wherein the calcium salt is administered over a period of at least about 10 minutes and the concentration of calcium is measured after administration of said calcium salt begins.

19. The method of claim 1, which further comprises limiting the subject's dietary intake of calcium for at least 12 hours prior to administering said calcium salt to the subject.

20. A method of detecting adynamic bone disease in a subject, said method comprising administering a calcium salt to the subject and measuring the level of calcium in the subject's blood at one or more time points after administration of said calcium salt begins,
and comparing the measured calcium level to a baseline level for the subject or to an average level for comparable subjects.

21. A method of detecting soft tissue calcification or a predisposition for soft tissue calcification in a subject, said method comprising administering a calcium salt to the subject and measuring the level of calcium in the subject's blood at one or more time points after administration of said calcium salt begins, and comparing the measured calcium level to a baseline level for the subject or to an average level for comparable subjects.

22. A method of detecting a predisposition for arteriosclerosis in a subject, said method comprising administering a calcium salt to the subject and measuring the level of calcium in the subject's blood at one or more time points after beginning the administration of said calcium salt,
and comparing the measured calcium level to a baseline level for the subject or to an average level for comparable subjects.

23. The method of claim 1, further comprising measuring the concentration of at least one other analyte in a bodily fluid from said subject.

24. The method of claim 23, wherein the at least one other analyte is phosphate.

25. The method of claim 23, wherein the at least one other analyte is magnesium.

26. The method of claim 23, wherein the at least one other analyte is parathyroid hormone or a fragment thereof.

27. A kit for detecting a calcium homeostasis disorder or a predisposition for such disorder, with the proviso that said disorder is not nephrolithiasis, osteoporosis, osteomalacia or primary hyperparathyroidism, said kit comprising:
a calcium salt,
means for collecting and storing samples of at least one bodily fluid for analysis, and
instructions, sample labels, and forms to enable a user to practice a method of the invention.

28. A method of doing business by providing analytical methods for detection of a calcium homeostasis disorder or a predisposition therefor, said method comprising:
providing a kit according to claim 27 to a user;
receiving at least one sample collected from a subject treated with a calcium salt from the user;
measuring the level of calcium in the at least one sample; and
communicating to the user the level of calcium in the samples.

29. The method of 28, further comprising measuring the level of at least one analyte other than calcium in the at least one sample;
and communicating to the user the level of at least one analyte other than calcium.
